(19) United States
(12) Patent Application Publication
(10) Pub. No.: US 2005/0244452 A1
(43) Pub. Date: Nov. 3, 2005

(54) Systems and methods for cell adhesion

(76) Inventor: Kenneth E. Gonsalves, Concord, NC (US)

Correspondence Address: J. Clinton Wimbish;Kilpatrick Stockton LLP, 1001 West Fourth Street, Winston-Salem, NC, 27101, US

(21) Appl. No.: 10/967,831

(22) Filed: Oct. 18, 2004

Related U.S. Application Data
(63) Continuation-in-part of application No. 10/835,757, filed on Apr. 30, 2004.

(51) Int. Cl. A61J 1/00; A61F 2/00

(52) U.S. Cl. 424/423; 427/2.14

(57) ABSTRACT

The present invention relates to the field of tissue engineering and biomaterials. In particular, the present invention provides systems and methods for biocompatible materials that promote cell adhesion while precluding the release of toxic or harmful substances from the materials. In an embodiment, the present invention provides a material comprising a polymeric layer comprising ions implanted therein, wherein the polymeric layer is biocompatible.

### PRIOR RELATED U.S. APPLICATION DATA

This application is a continuation-in-part of U.S. patent application Ser. No. 10/835,757 filed Apr. 30, 2004, which is hereby incorporated by reference in its entirety.

### STATEMENT OF GOVERNMENT LICENSE RIGHTS

This invention was sponsored in part through the support of the North Carolina Biotechnology Center Grant # 2002-IDG-1016.

### TECHNICAL FIELD OF THE INVENTION

The present invention relates to the field of tissue engineering and biomaterials. In particular, the present invention relates to biomaterials operable to promote cell adhesion.

### BACKGROUND OF THE INVENTION

Connective tissues are responsible for providing and maintaining form in the body. Functioning in a mechanical role, they provide a matrix that connects and binds cells and organs and ultimately gives support to the body. Unlike other tissue types (epithelium, muscle, and nerve), which are formed mainly by cells, the major constituent of connective tissue is its extracellular matrix, composed of protein fibers, ground substance, and tissue fluid. Embedded within the extracellular matrix are the connective tissue cells.

Structurally, connective tissue can be divided into three classes of components: cells, fibers, and ground substance. The wide variety of connective tissue types in the body reflects variations in the composition and amount of these components, which are responsible for the remarkable structural, functional, and pathologic diversity of connective tissue.

Connective tissue serves a variety of functions, the most conspicuous being structural. The capsules that surround the organs of the body and the internal architecture that supports their cells are composed of connective tissue. This tissue additionally constitutes tendons, ligaments, and areolar tissue that fills spaces between organs. Bone, cartilage, and adipose tissue are specialized types of connective tissue that support the soft tissues of the body and store fat.

As a result of its structural importance in the body, connective tissue is a heavily researched area for tissue engineering replacement alternatives. According to the National Center of Health Statistics, for example, greater than two billion dollars are spent annually in the United States on bone related implants comprising hip replacements, knee replacements, dental implants, and pins to stabilize or repair fractures. A common class of implants for stabilization, repair, and regeneration of connective tissue, especially bone, comprise metals. Metal implants find wide applicability primarily due to their ability to bear significant loads, withstand fatigue loading, and undergo plastic deformation prior to failure.

Along with the advantages of metal implants come several disadvantages. A significant disadvantage of metal implants is that the interaction between the connective tissue, such as bone, and the implant does not comprise a chemical bond. The lack of chemical bonding between the metal implant and the connective tissue may compromise the fixation of the implant, which may result in an attendant loosening of the implant over a period of time. Mechanical loosening of implants from the connective tissue can result in excessive joint displacement and generally mandates the need for revision surgery, which is more difficult, less successful, causes additional damage to surrounding tissues, and is economically frustrating. Another significant disadvantage of metal implants is their tendency to experience corrosion and release metallic ions. Metallic ions released from an implant may act as allergens and/or toxic sources due to their know adverse effects on human cells. A further disadvantage of metallic implants is that their high modulus limits their applicability to environments where greater degrees of freedom are commonly encountered.

In light of the disadvantages of metal implants, it would be advantageous to provide biocompatible materials for applications that promote the adhesion of connective tissues while precluding the release of harmful or toxic substances such as metal ions. It would be additionally advantageous to provide biocompatible materials that may be tailored to display a modulus commensurate with their physiological environment.

### SUMMARY OF THE INVENTION

The present invention provides systems and methods for biocompatible materials that promote cell adhesion while precluding the release of toxic or harmful substances from the materials. In one embodiment, the present invention provides a material comprising a polymeric layer comprising ions implanted therein, wherein the polymeric layer is biocompatible.

In another embodiment, the present invention provides a lithographic process comprising exposing a recording medium to an ion source to form a pattern, wherein the lithographic recording medium comprises a biocompatible polymer.

In another embodiment, the present invention provides an apparatus comprising a substrate, and a polymeric substrate coating comprising ions implanted therein, wherein the polymeric substrate coating is biocompatible. In some embodiments, the substrate may comprise a metal or alloy, a polymeric material, or a combination thereof. In embodiments of the present invention, the apparatus may be used as an implant.

In a further embodiment, the present invention provides a biocompatible polymer comprising at least one phosphorus containing monomer and a cyclic component comprising at least one cyclic containing monomer operable to undergo ring opening polymerization, wherein the biocompatible polymer is biodegradable.

In a still further embodiment, the present invention provides an apparatus comprising a biocompatible polymer comprising a phosphorus component comprising at least one phosphorus containing monomer and a cyclic component comprising at least one cyclic containing monomer operable to undergo ring opening polymerization, wherein the biocompatible polymer is biodegradable. In embodiments of the present invention, the apparatus may be utilized as an implant.

A feature and advantage of the present invention is that, in an embodiment, the present invention provides materials that may facilitate or promote cell adhesion while precluding the release of toxic substances from the material.

Another feature and advantage of the present invention is that, in an embodiment, the present invention provides an apparatus that may be tailored to reflect the modulus of the physiological environment in which it is placed.

### BRIEF DESCRIPTION OF THE FIGURES

FIG. 1 illustrates a biocompatible polymeric layer according to an embodiment of the present invention.

FIG. 2 illustrates a lithographic process according to an embodiment of the present invention.

FIG. 3A illustrates a biocompatible polymeric layer according to an embodiment of the present invention.

FIG. 3B illustrates a cross-sectional analysis of a biocompatible polymeric layer according to an embodiment of the present invention.

FIG. 4A illustrates a biocompatible polymeric layer according to an embodiment of the present invention.

FIG. 4B illustrates a cross-sectional analysis of a biocompatible polymeric layer according to an embodiment of the present invention.

FIG. 5 illustrates x-ray photoelectron emission from a biocompatible polymeric layer according to an embodiment of the present invention.

FIG. 6 illustrates x-ray photoelectron emission from a biocompatible polymeric layer according to an embodiment of the present invention.

FIG. 7 illustrates secondary ion mass spectroscopy data for a biocompatible polymeric layer according to an embodiment of the present invention.

### DETAILED DESCRIPTION OF THE INVENTION

The present invention provides systems and methods for biocompatible materials that promote cell adhesion while precluding the release of harmful or toxic substances from the materials. The biocompatible materials of the present invention may be used in implants and may be tailored to display a modulus commensurate with their physiological environment.

Reference is made below to specific embodiments of the present invention. Each embodiment is provided by way of explanation of the invention, not as a limitation of the invention. In fact, it will be apparent to those skilled in the art that various modifications and variations can be made in the present invention without departing from the scope or spirit of the invention. For instance, features illustrated or described as part of one embodiment may be incorporated into another embodiment to yield a further embodiment. Thus, it is intended that the present invention cover such modifications and variations as come within the scope of the appended claims and their equivalents.

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary, depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein, and every number between the end points. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10, as well as all ranges beginning and ending within the end points, e.g., 2 to 9, 3 to 8, 3.2 to 9.3, 4 to 7, and finally to each number 1, 2, 3, 4, 5, 6, 7, 8, 9 and 10 contained within the range. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent.

In one embodiment, the present invention provides a material comprising a polymeric layer comprising ions implanted therein, wherein the polymeric layer is biocompatible. The polymeric layer may comprise poly(methyl methacrylate), polyphosphazenes, polymers formed from phosphorus-containing cyclic esters, polysiloxanes, polyethylene, or any copolymeric combination thereof. The polymeric layer may additionally comprise any other biocompatible polymer known to one of ordinary skill in the art.

In embodiments where the polymeric layer comprises polymers formed from phosphorus-containing cyclic esters, the phosphorus-containing cyclic esters may comprise cyclic phosphates of the general formula: 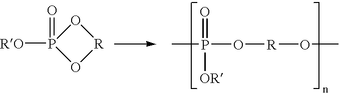
wherein R and R′ may comprise aliphatic groups. Alternatively, the phosphorus-containing cyclic esters may comprise cyclic phosphonates of the general formula: 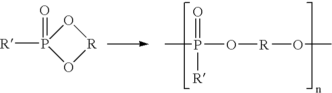
wherein R and R′ comprise aliphatic groups.

In an embodiment where the polymeric layer comprises polyethylene, the polyethylene may comprise ultra-high molecular weight polyethylene.

The biocompatible polymeric layer comprises ions implanted therein. Ions suitable for implantation into the polymeric layer may comprise calcium ions, phosphorus ions, magnesium ions, potassium ions, sodium ions, argon ions, or any combination thereof. The ions may be implanted into the polymeric layer by irradiation of the polymeric layer with an ion source such as a general purpose Cockcroft-Walton-type ion implanter with a modified Freeman source. The ions implanted in the polymeric layer may penetrate the polymeric layer to a depth of less than about 1 μm.

In an embodiment of the present invention, the polymeric layer comprising ions implanted therein may display a thickness of at least about 10 μm. In some embodiments, the polymeric layer may comprise a thickness of at least about 5 μm. In other embodiments, the polymeric layer may comprise a thickness of at least about 1 μm. In a further embodiment, the polymeric layer may comprise a thickness ranging from about 1 μm to about 10 μm. In a still further embodiment, the polymeric layer may comprise a thickness less than about 1 μm.

Irradiation of the polymeric layer with energetic ions according to embodiments of the present invention may lead to dramatic modification of the polymer surfaces. While not wishing to be bound by any theory, the ions penetrate the surface and may create significant changes by interacting with the polymer atoms via electronic (ionization) and nuclear (recoil) interactions. See, for example, P. K. Chu et al., Mater. Sci. Eng. R. 36(5-6), 143-206 (2002). Irradiation of the polymer layer with ions may produce cavities within the polymeric layer where the ions strike the surface of the layer.

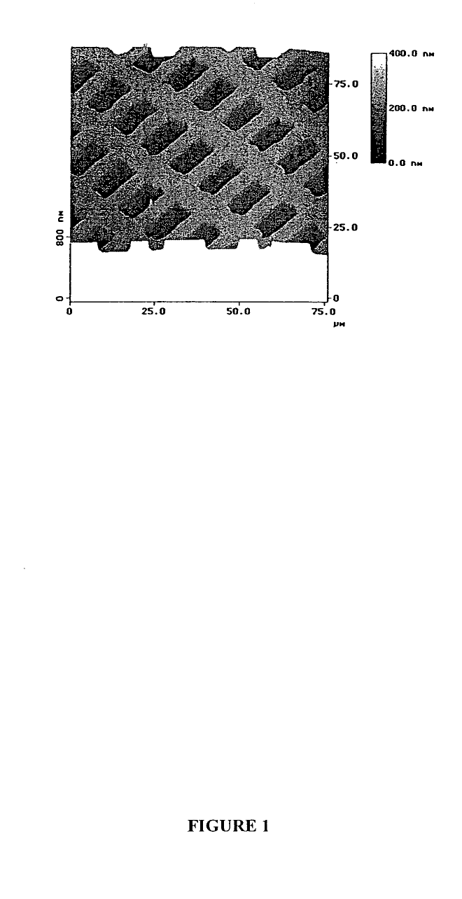

Referring now to the figures wherein the like numerals indicate like elements throughout the several figures, FIG. 1 illustrates a polymeric layer comprising ions implanted therein according to an embodiment of the present invention. FIG. 1 displays an atomic force microscopy (AFM) of a poly(methyl methacrylate) layer after exposure with 85 keV, 1×10¹⁵ ions/cm² P⁺ ions, recorded in tapping mode. Prior to irradiation with the ions source, the poly(methyl methacrylate) layer was covered with a mask to pattern the exposure of the layer to the ions. As demonstrated in FIG. 1, the irradiation of the poly(methyl methacrylate) layer with phosphorus ions produced cavities in the layer corresponding to points of phosphorus ion penetration. The formation of cavities in the polymeric layer increases the roughness of the polymeric layer.

The ions implanted in the polymeric layer in conjunction with the enhanced roughness of the layer may facilitate and/or promote cell adhesion to the polymeric layer. The implanted ions may promote cellular adhesion by leading to the formation of various compounds and structures that facilitate cellular interaction with the polymeric layer.

In embodiments of the present invention, the various compounds and structures formed by ion implantation may mimic the physiological extracellular matrix produced connective tissue cells. In the formation of bone, for example, osteoblast cells are responsible for the synthesis and deposition of the organic and inorganic components of the bone matrix. During bone matrix synthesis, osteoblasts have the ultrastructure of cells actively synthesizing proteins for export. Osteoblasts are polarized cells. Matrix components are secreted at the cell surface, which is in contact with the older bone matrix. Inorganic matter represents about 50% of the dry weight of bone matrix. Calcium and phosphorus are especially abundant, but bicarbonate, citrate, magnesium, potassium, and sodium are additionally found. X-ray diffractions studies have shown that calcium and phosphorus form hydroxyapatite crystals with the compositions Ca₁₀(PO₄)₆(OH)₂. Significant quantities of amorphous calcium phosphate are also present.

While not wishing to be bound by any theory, the implantation of phosphorus and calcium ions into polymeric layers of the present invention may promote the formation of calcium phosphates and hydroxyapatite structures that may facilitate and/or promote interaction of osteoblast cells with the polymeric layer. The interaction between the osteoblast cells and the ion implanted polymeric layer is chemical in nature as osteoblasts are polarized cells making them amenable to electrostatic interactions such as hydrogen bonding.

Moreover, polymeric layers comprising ions implanted therein may facilitate and/or promote the interaction and adhesion of other connective tissue cells such as chondroblast cells and fibroblast cells in a manner similar to that previously described for osteoblast cells.

In another embodiment, the present invention provides a lithographic process for constructing a polymeric layer comprising ions implanted therein, wherein the polymeric layer is biocompatible. In one embodiment, the lithographic process comprises exposing a lithographic recording medium to an ion source to form a pattern, wherein the lithographic recording medium comprises a biocompatible polymer. Recording media suitable for use with the present lithographic process may comprise polymeric layers consistent with those previously described. The recording media may comprise poly(methyl methacrylate), polyphosphazenes, polymers formed from phosphorus-containing cyclic esters, polysiloxanes, polyethylene, ultra-high molecular weight polyethylene, or any copolymeric combination thereof.

Ion sources suitable for use with the present invention may comprise those operable to provide calcium ions, phosphorus ions, magnesium ions, potassium ions, sodium ions, and/or argon ions.

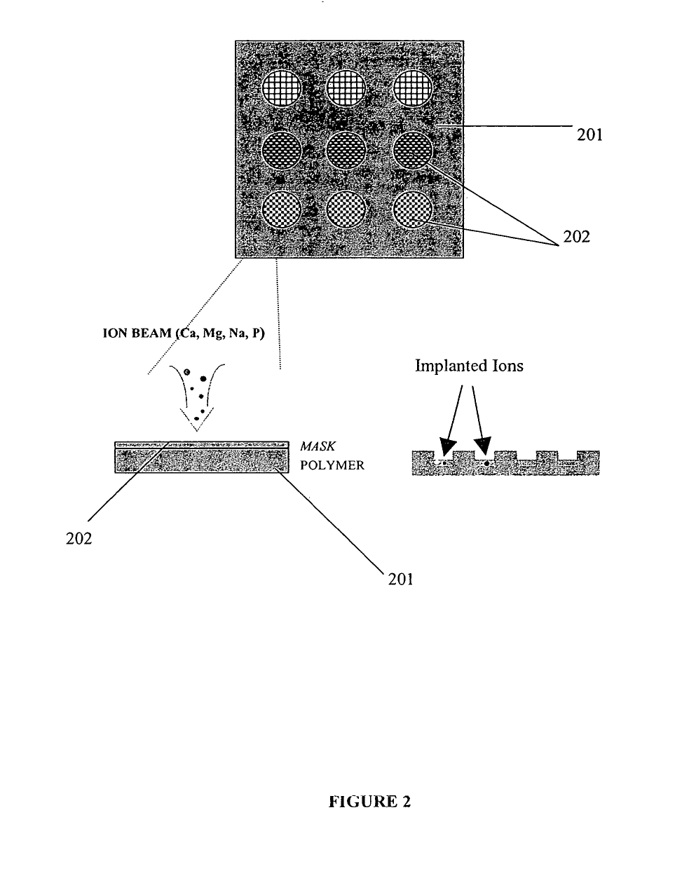

FIG. 2 illustrates a lithographic process according to an embodiment of the present invention. According to FIG. 2, a polymeric layer 201 may be covered with a mask 202 to delineate a pattern on the surface of the polymer layer. The mask placed on the polymeric layer is resistant to the penetration of ions. After masking, the polymeric layer is exposed to an ion source comprising an ions collimated in a beam. The calcium ions, phosphorus ions, magnesium ions, potassium ions, sodium ions, argon ions, or combinations thereof in the ion beam penetrate the polymeric layer forming nanostructures in the polymeric layer at the impact points.

In another embodiment, the present invention provides an apparatus comprising a substrate and a polymeric substrate coating comprising ions implanted therein, wherein the substrate coating is biocompatible. In some embodiments of the present invention, the substrate may comprise a metal or alloy. Metals and/or alloys suitable for use with the present invention may comprise titanium, cobalt, chromium, molybdenum, nickel, and/or stainless steel. In other embodiments of the present invention, the substrate may comprise a polymeric material such as polyethylene, ultra-high molecular weight polyethylene, polypropylene, or polycarbonate. In a further embodiment, the substrate may comprise a metal or alloy inner layer and a polymeric outer layer. Alternatively, the substrate may comprise a polymeric inner layer and a metal or alloy outer layer.

The polymeric substrate coating may comprise a polymeric layer consistent with the ion-implanted polymeric layer previously described. The polymeric substrate coating may comprise poly(methyl methacrylate), polyphosphazenes, polymers formed from phosphorus-containing cyclic esters, polysiloxanes, polyethylene, ultra-high molecular weight polyethylene, or any copolymeric combination thereof. The polymeric layer may additionally comprise any other biocompatible polymer known to one of ordinary skill in the art. Moreover, the ions implanted in the polymeric layer may comprise calcium ions, phosphorus ions, magnesium ions, potassium ions, sodium ions, argon ions, or any combination thereof.

In embodiments of the present invention, the polymeric substrate coating comprising ions implanted therein may facilitate and/or promote the adhesion of tissue cells to the coating. The polymeric substrate coating may facilitate and/or promote cell adhesion in a manner consistent with that for previously described polymeric layer comprising ions implanted therein. The irradiation of polymeric substrate coating with an ion source, for example, may produce various compounds and structures that mimic the physiological extracellular matrix produced by connective tissue cells. The present polymeric substrate coating may facilitate and/or promote the adhesion of osteoblast cells, chondroblast cells, and/or fibroblast cells.

In being operable to facilitate and/or promote the adhesion of connective tissue cells through a polymeric substrate coating, the present apparatus may be utilized as an implant for various medical purposes. The apparatus may be used, for example, as an implant to repair damaged bone or cartilage.

Moreover, the compositional structure of the present apparatus comprising a substrate and a polymeric substrate coating allows tailoring of the apparatus to reflect the modulus of the physiological environment in which it is to reside as an implant. Modulus is defined as the resistance to deformation as measured by the initial stress divided by ΔL/L wherein L is defined as length. An environment with a high modulus demonstrates very little ΔL under stress and is, therefore, classified as rigid. An environment with a low modulus, on the other hand, displays a large ΔL and is classified as elastomeric.

Physiological environments within human and animal bodies display varying moduli. The physiological environment surrounding a joint such as an elbow or knee, for example, may display a low modulus due to the increased degrees of freedom. Moreover, the physiological environment surrounding the central portion of a femur may display low modulus due to restricted degrees of freedom.

In embodiments, the apparatus of the present invention comprising a substrate and polymeric substrate coating may be tailored to reflect the modulus of the physiological environment in which it is to reside. If a high modulus is desired, a substrate with a high modulus may be chosen. A metal or alloy, for example, may be chosen due to the inherent rigidity of metals or alloys. Rigid polymeric materials may also be chosen for environments necessitating a high modulus, such as ultra-high molecular weight polyethylene, polypropylene, and polycarbonate. Alternatively, if a lower modulus is desired, a more elastomeric polymeric material may be chosen for the substrate. In some embodiments, metals or alloys may be combined with polymeric materials to arrive at an appropriate modulus for a particular application. Once the proper substrate is chosen for a particular application, the polymeric substrate coating may be applied to facilitate and/or promote cell adhesion to the apparatus. The polymeric layer may be applied to the substrate by any method known to one of ordinary skill in the art such as spin-coating. In an embodiment, the substrate may be initially processed to enhance interaction with the polymeric substrate coating. The substrate, for example, may be coated with a polymeric material operable to facilitate binding of the polymeric substrate coating to the substrate. Alternatively, the substrate may be etched to promote adhesion of the polymeric substrate coating to the substrate.

Once the polymeric substrate coating is applied to the substrate, the polymeric substrate coating may be irradiated with an ion source to implant ions within the coating. The previously described lithographic process may be used to achieve the desired ion implantation into the polymeric substrate coating.

The ability to tailor the modulus of the present apparatus may reduce the mechanical stress placed on the apparatus when utilized as an implant in a human or animal body. The reduced mechanical stress on the apparatus in conjunction with the polymeric substrate coating may further facilitate and/or promote the adhesion of tissue cells, such as osteoblast, chondroblast and/or fibroblast cells, to the implant. The increased interaction between the implant and tissue cells may result in an attendant reduction in mechanical loosening of the implant as well as a reduction in implant failure.

In addition to enhancing the mechanical properties of an implant, the present apparatus comprising a substrate and a polymeric substrate coating may preclude the release of harmful substances, such as metallic ions, from the implant into the body. As previously discussed, a significant problem with metallic implants is their potential to release metallic ions into the body. In embodiments of the present invention where a metal or alloy substrate is utilized, the polymeric substrate coating may diminish and/or preclude the release of metallic ions from the substrate into the body of a patient. The polymeric substrate coating may be constructed to a thickness sufficient to minimize or preclude the release of metallic ions from the substrate. In some embodiments, the metal or alloy substrate may be coated with an intermediate polymeric layer before application of the polymeric substrate coating. The intermediate polymeric layer may be operable to further prevent the release of harmful or toxic substances into the body from the implant. In such embodiments, the intermediate polymeric layer may be constructed to a thickness sufficient to minimize and/or preclude the release of metallic ions from the substrate. In other embodiments, the use of a high modulus polymer in place of a metal or alloy as the substrate removes the potential release of metallic ions from the substrate into the body altogether. As a result, in embodiments, the present apparatus may be used to provide implants with enhanced mechanical properties and reduced toxicological effects.

In another embodiment, the present invention provides a biocompatible polymer comprising a phosphorus component comprising at least one phosphorus containing monomer and a cyclic component comprising at least one cyclic containing monomer operable to undergo ring opening polymerization wherein the biocompatible polymer is biodegradable. In one embodiment, the phosphorus component of the present biocompatible polymer may comprise dimethylphosphonate (VPE), vinylphosphonic acid (VPA), or any combination thereof.

The cyclic component of the present biocompatible polymer may comprise lactones, lactams, cyclic acetals, phosphorus-containing cyclic esters, epoxides, or any combination thereof. In one embodiment, the cyclic component may comprise ε-caprolactone, δ-valerolactone, 3-propanolactam, 4-butanolactam, 5-pentanolactam, 6-hexanolactam, 1,3,5-trioxepane, 1,3,5-trioxane, 1,3,6,9-tetraoxacycloundecane, or 1,3-dioxacycloalkanes such as 1,3 dioxolane, 1,3-dioxepane, and/or 1,3-dioxocane.

The present biocompatible polymer comprising a phosphorus component and cyclic component may facilitate and/or promote the adhesion of connective tissue cells. While not wishing to be bound by any theory, in some aspects the biocompatible polymer may simulate the chemical environment of the extracellular matrices of the connective tissue cells. In the case of bone, for example, the presence of phosphorus moieties in the polymer may lead to the formation of calcium phosphates and hydroxyapatite structures that may facilitate and/or promote the interaction of osteoblast cells with the polymer. In addition to osteoblast cells, the biocompatible polymer may facilitate and/or promote the adhesion of chondroblast cells and/or fibroblast cells.

In one embodiment, the present biocompatible polymer comprising a phosphorus component and a cyclic component is biodegradable. The polymer may be operable to degrade under the physiological conditions present in human or animal bodies.

In another embodiment, the biocompatible polymer comprising a phosphorus component and a cyclic component may further comprise ions implanted therein. The present polymer may be irradiated with an ion source in a manner consistent with that previously described for other polymeric materials according to embodiments of the present invention. Ions suitable for implantation into the polymer may comprise calcium ions, phosphorus ions, magnesium ions, potassium ions, sodium ions, argon ions, or any combination thereof.

The implantation of ions into the polymeric layer comprising a phosphorus component and a cyclic component may form inorganic nanostructures in the polymeric layer which may further facilitate and/or promote the adhesion of tissue cells such as osteoblasts, chondroblasts, and/or fibroblasts. Calcium phosphates and hydroxyapatite structure formation, for example, may be facilitated by ion implantation into the polymer.

In another embodiment, the present invention provides an apparatus comprising a biocompatible polymer comprising a phosphorus component comprising at least one phosphorus containing monomer, and a cyclic component comprising at least one cyclic containing monomer operable to undergo ring opening polymerization, wherein the biocompatible polymer is biodegradable.

The apparatus may comprise a biodegradable polymer consistent with the one previously described. In one embodiment, the phosphorus component of the polymer of the present apparatus may comprise dimethylphosphonate (VPE), vinylphosphonic acid (VPA), or any combination thereof.

The cyclic component of the polymer of the present apparatus may comprise lactones, lactams, cyclic acetals, phosphorus-containing cyclic esters, epoxides, or any combination thereof. In one embodiment, the cyclic component may comprise ε-caprolactone, δ-valerolactone, 3-propanolactam, 4-butanolactam, 5-pentanolactam, 6 hexanolactam, 1,3,5-trioxepane, 1,3,5-trioxane, 1,3,6,9-tetraoxacycloundecane, or 1,3-dioxacycloalkanes such as 1,3 dioxolane, 1,3-dioxepane, and/or 1,3-dioxocane.

In one embodiment, the polymer of the present apparatus comprises ions implanted therein. Ions suitable for implantation into the polymer of the present apparatus may comprise calcium ions, phosphorus ions, magnesium ions, potassium ions, sodium ions, argon ions, or any combination thereof.

In an embodiment of the present invention, the present apparatus comprising a biocompatible polymer comprising a phosphorus component and a cyclic component may be used as a scaffold or implant for the repair and/or regeneration of connective tissue. The biocompatibility and biodegradability of the polymer renders the present apparatus suitable for serving as a scaffold or implant. In embodiments, the present apparatus may facilitate and/or promote the adhesion of connective tissue cells such as osteoblast cells, chondroblast cells, and/or fibroblast cells.

### EXAMPLES

### Examples of a Polymeric Layer Comprising Ions Implanted Therein

A polymeric layer comprising poly(methyl methacrylate) (PMMA) was formed by spin-casting a PMMA solution on a silicon wafer. The polymeric layer was 217 nm thick as measured by a Tencor Alphastep 200 surface profilometer. After polymeric layer formation, a fine nickel mesh obtained from Buckbee-Mears, St. Paul, Minn., was placed on the newly form polymeric layer to serve as a mask. The mask had a maximum transmittance of 36% and the space between the wires was 7.62 μm. A piece of the mesh measuring 0.7 in.×0.7 in. was placed on the poly(methyl methacrylate) film using copper tape.

Phosphorus ions (P⁺) were applied to the polymeric layer using an Extrion implant accelerator, a general purpose Cockcroft-Walton-type ion implanter with a modified Freeman source. The implants were performed by raster scanning the ion beam over a circular implant area of about 4 cm² thereby assuring a uniform implanted dose over the entire ion implant area. The sample was clamped to a sample holder that was maintained near room temperature. The sample holder was biased to +67V to suppress secondary electron emission, and was surrounded by a Faraday cage at −300V for both secondary electron suppression and secondary ion collection. The absolute accuracy with suppression is generally better than 10%. However, the linearity is much better, usually better than 1%. The sample current was measured as the sum of the current on the sample holder and the suppressor. P⁺ ion implantation was carried out at an energy of 85 keV with ion fluences of 1×10¹⁵ ions/cm².

After exposure to the ion beam for phosphorus ion implantation, the meshes were removed and the PMMA layer was characterized using the following methods:
Atomic force microscopy (AFM): Surface morphology examinations were conducted by AFM. Imaging was performed at room temperature using a commercial optical lever microscope (Nanoscope III, Digital Instruments). Standard-geometry silicon nitride probes (TESP) tips 125 μm in length and with a typical frequency between 294 and 375 kHz were used (Digital Instruments). Tapping mode topographic images were taken in air in the constant deflection mode, with a very slow scan rate of 1 Hz which provided less contact between the AFM tip and the imaged sample, leaving the sample surface in its intact mode.
X-ray photoelectron spectroscopy (XPS) investigation was performed with a Riber LAS-3000 system. Electron ejection from the samples was induced by 12 kV×15 mA Mg K_{α} X-ray radiation at a pass energy of 20 eV and a step size of 0.1 eV. The pressure in the sample chamber was kept below 1×10⁻⁹ Torr.

Charging of the samples, due to photoemission, was corrected by setting the energy of the main hydrocarbon component of C₁ₛ spectra at 285.0 eV.
Dynamic secondary ion mass spectroscopy (SIMS) measurement was carried out using a PHI Quadrupole SIMS instrument (Physical Electronics, Inc.) with a cesium primary beam at an impact energy of 3 keV. The primary ion angle of incidence was 60°. Charge neutralization was applied.

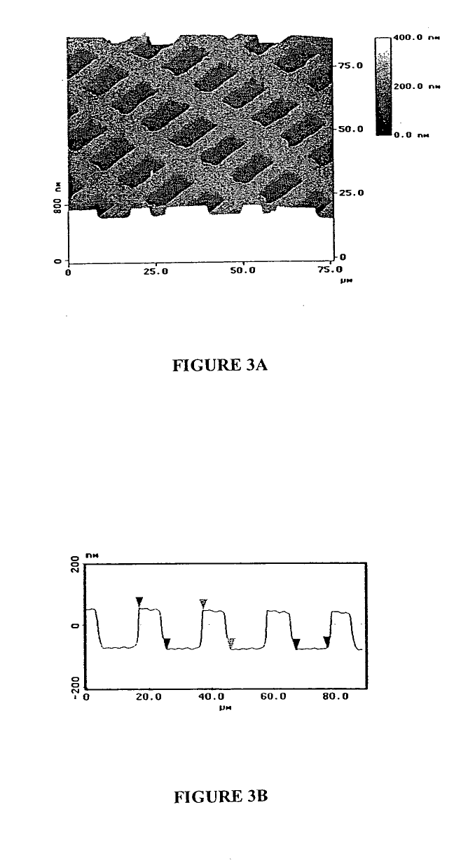

FIG. 3A displays an AFM image of the PMMA layer after exposure with 85 keV, 1×10¹⁵ ions/cm² P⁺ ions recorded in tapping mode with typical surface features characterized by a cross-sectional analysis. The AFM results of the cross-section analysis of the P⁺ ions irradiated PMMA sample, as illustrated in FIG. 3B, displayed a distance between isolated islands of about 9.8 μm and an island height of about 129 nm. It can be seen from the cross-sectional analysis that the cavity shape produced by ion implantation is conical in nature. It was additionally observed that the walls around the cavities were not symmetrical on all sides. Along the shorter sides of the rectangular cavities, the walls are about 40% thicker than in the other directions.

For comparative purposes, a PMMA layer comprising argon ions (Ar⁺) implanted therein was additionally prepared. Prior to argon ion implantation, the PMMA layer was prepared in a manner identical to that of the previously described PMMA layer comprising implanted phosphorus ions. The conditions used for argon ion implantation were chosen based on calculation from a TRIM program such that the resulting projection range would be similar to that achieved in the P⁺ implantations. The argon implanted PMMA layer was characterized in a manner consistent with the characterization of the phosphorus ion implanted PMMA layer.

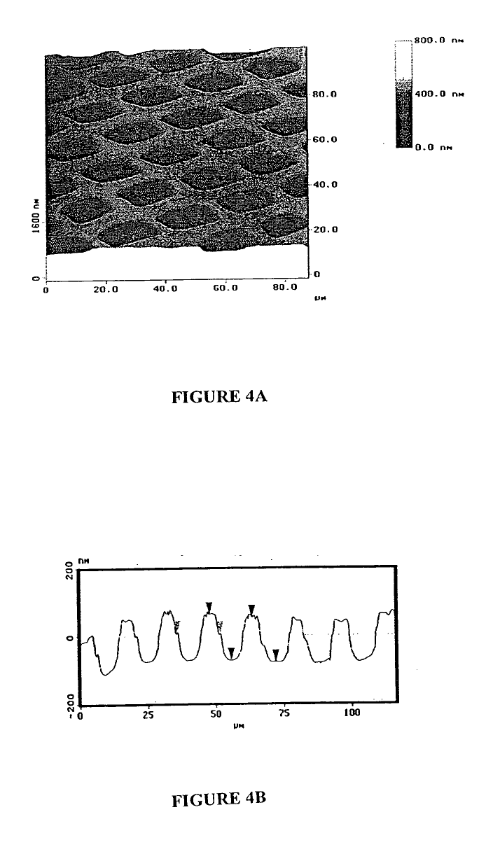

The experimental results were in good agreement with the prediction. As demonstrated in FIG. 4A, arrays of cavities were observed uniformly distributed on the PMMA surface that had been exposed to 115 keV, 1×10¹⁵ ions/cm² Ar⁺ ions. Similar to the patterns created after exposure of P⁺ ions (FIG. 3), the walls around the wells were not the same on all sides. Cross section analysis with AFM, as illustrated in FIG. 4B, showed that the distance between the islands was 11.5 μm, and the depth 133 nm.

It has been observed that the effect of ion implantation on the material is confined to a very thin layer beneath the surface, usually less than a micrometer (Chu et al., 2002). Therefore, nano-size features can be achieved by properly selecting the energy of the ion beam. Besides surface profile, surface roughness of each sample was also determined in the AFM studies by measuring the root mean square roughness (Rᵣₘₛ). Rᵣₘₛ of the PMMA surfaces after exposure to ion implantations was around 60 nm. As expected from the TRIM program, similar rough surfaces were achieved for both P⁺ and Ar⁺ ion implantations. The PMMA surface exposed to P⁺ ions was a little bit rougher in contrast to the Ar⁺ ions irradiation. This finding could be explained by a larger effect of the heavier Ar⁺ on the PMMA chemical structure, which in turn may lead to a decrease of free volume fraction in the PMMA surface layer and subsequent densification and compaction. See \check{S}vor\check{c}ík et al., J. Mater. Sci.: Mater. Med. 11, 655-660 (2000).

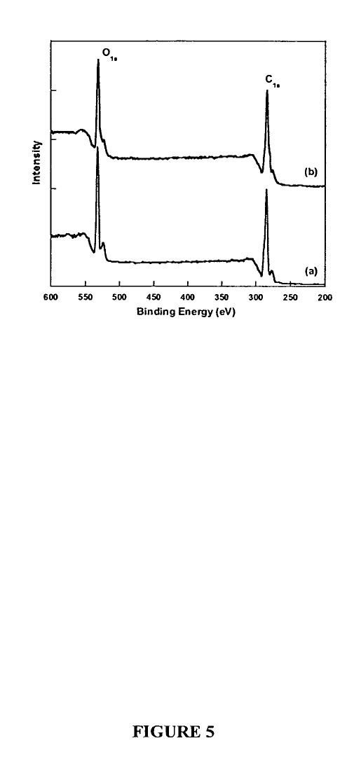

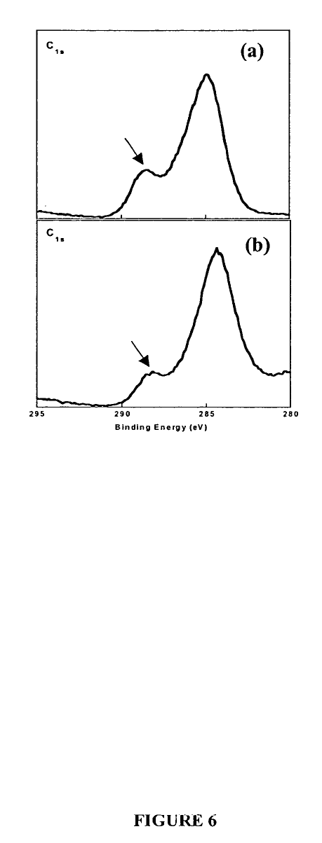

To study how ion implantation affects the chemical properties of the substrate, surface chemical state comparison using X-ray photoelectron spectroscopy (XPS) was conducted to reveal the difference between the pristine and P⁺ implanted PMMA samples with the fluence of 1×10¹⁵ ions/cm². FIG. 5 illustrates characteristic C_{1S} (285 eV) and O₁ₛ (535 eV) XPS signals. It can be seen in the XPS survey spectra obtained under low spectral resolution conditions that the O₁ₛ peak relative intensity decreases in going from the pristine (spectrum a) to the P⁺ ions implanted sample (spectrum b). The change in the relative amount of oxygen is caused by the loss of the O-containing pendant methylester groups. To further prove the cleavage of some pendant groups, C₁ₛ signals were measured. As seen in FIG. 6, in going from the pristine (spectrum a) to the P⁺ ions implanted sample (spectrum b), the decrease of the 288.5 eV component of the C₁ₛ peak, which is associated with carbons of the O—C═O groups, testifies to the destruction of the methylester groups from the polymer backbone.

In order to evaluate the influence of the ion beam treatments on cell adhesion, mouse calvaria osteoblast cells were seeded on the unirradiated and irradiated P⁺ and Ar⁺ PMMA surfaces. Normal osteoblast cell cultures were prepared from mouse neonates according to a method previously described for chick embryos. See Ramp et al., Bone Miner. 24, 59-73 (1994). Bone-forming cells were isolated from mouse neonate calvariae by sequential collagenase-protease digestion. The isolated cells were pooled in mouse osteoblast growth medium (OBGM) consisting of Dulbecco's modified Eagle's medium with 25 mM HEPES, 10% fetal bovine serum, 2 g/L sodium bicarbonate, 75 μg/ml glycine, 100 μg/ml ascorbic acid, 40 ng/ml vitamin B₁₂, 2 μg/ml p-aminobenzoic acid, 200 ng/ml biotin, and 100 U/ml-100 μg/ml-0.25 μg/ml penicillin-streptomycin-fungizone (pH 7.4). See Ramp et al., Bone Miner., 15, 1-17 (1991). Cells were then seeded into 25 cm³ flask at a density of 10⁶ osteoblasts/flask and incubated at 37° C. in a 5% CO₂ atmosphere until they reached approximately 80% confluency. Ostocalcin, type I collagen, and alkaline phosphatase were selected to characterize isolated mouse osteoblasts. Measurement of osteoblast attachment to the various surfaces was performed essentially as previously described. See Dalton et al., Bio Techniques, 21, 298-303 (1996). Media was removed from flasks containing osteoblasts, and the osteoblasts were rinsed with Hank's balanced salt solution (HBSS). Osteoblasts were then metabolically-labeled by culturing for 18 h in OBGM labeling medium containing methionine-free Dulbecco's modified Eagle's medium supplemented with [³⁵S] methionine (Translabel 51006; ICN Biomedicals, Costa Mesa, Calif., USA) at a concentration of 0.185 MBq/mL (5 μCi/ml). Following the 18 h labeling period, media was removed from osteoblast culture, and osteoblasts were rinsed with HBSS. Osteoblast cells were detached, resuspended, and seeded into 6-well cluster plate.

PMMA samples irradiated with P⁺ and Ar⁺ ions were placed in the well. Pristine PMMA was used as control. The seeding density was 150,000 cells per well. After incubation at 37° C. in a 5% CO₂ atmosphere for 24 h, the culture plate was rinsed three times with HBSS. The plate was then allowed to air dry. Samples were exposed to a Kodak storage phosphor screen (SO₂₃₀; Molecular Dynamics, Sunnyvale, Calif., USA) for 2 h, and protected from light during that time. The screen was then scanned in a Typhoon 8600 Variable Mode PhosphorImager (Molecular Dynamics), which converts regions of higher energy in the screen to a digital image, in which pixel values are equivalent to energy levels. Using ImageQuant software (version 5.2) (Molecular Dynamics) a grid was created and superimposed over the area representative of each wafer as previously described. See Dalton et al., (1996). An ImageQuant program was used to quantify the pixel values in each grid (as described in ImageQuant Users Guide). The results were the mean and standard deviation of pixel values.

As shown in Table 1, significant differences were observed in osteoblastic cells' responses to PMMA exposed to ion irradiation. Both P⁺ ions implanted and Ar⁺ ions implanted PMMA samples have more cells attached than the untreated regular PMMA, indicating that ion implantation does improve osteoblast adhesion on polymeric substrate, due to the increased surface roughness. This is consistent with what Webster and coworkers found in their studies, where strong correlations between increased surface roughness and enhanced osteoblast adhesion was demonstrated. See Webster et al., Scripta Mater. 44(8/9), 1639-1642 (2001), and references therein. Although similar surface topography and surface roughness were observed for P⁺ irradiated and Ar⁺ irradiated PMMA films, the former has more cells attached than the

TABLE 1
surface roughness and the relative amount of osteoblast cells (R.O.)
attached after 24 hours.

| | ENERGY | DOSAGE | | |
|---|---|---|---|---|
| IONS | (KEV) | (IONS/CM²) | R_{RMS} | R.O. |
| P | 85 | 1 × 10¹⁵ | 60.525 | 2.44 |
| Ar | 115 | 1 × 10¹⁵ | 57.613 | 1.72 |
| No Ions | N/A | N/A | — | 1.00 |

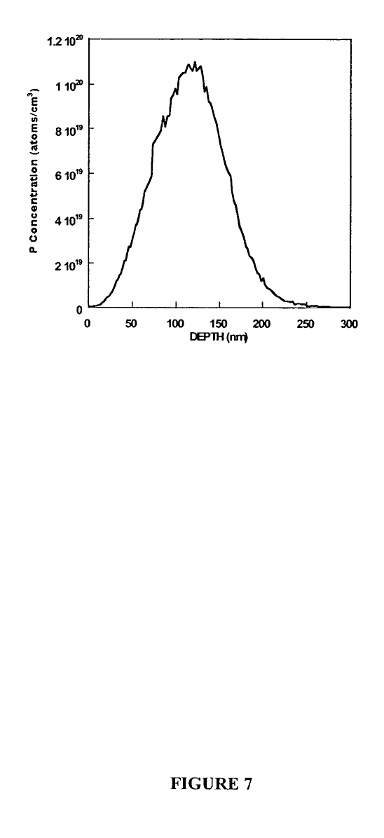

latter, implying that surface morphology is not the only factor that promoted osteoblast adhesion. Selection of ion species also is important for cell adhesion. Ar⁺ ions are inert, but P⁺ ions implanted to the polymeric substrate might have helped improve osteoblast adhesion. The distribution of P⁺ ions on the PMMA film was determined by secondary ion mass spectroscopy (SIMS). Shown in FIG. 7 is the dynamic SIMS depth profiling data for P⁺ in the treated PMMA film. The concentration of P ions versus depth was displayed, and the majority of P ions are distributed in the area that is about 100 nm from the surface. The maximum amount of P ions, 1.1×10²⁰/cm³ was found at 121 nm.

### Example of a Biocompatible Polymer Comprising a Phosphorus Component and Cyclic Component

A biocompatible polymer comprising a phosphorus component comprising at least one phosphorus containing monomer and a cyclic component comprising at least one cyclic containing monomer operable to undergo ring-opening polymerization wherein the polymer is biodegradable was constructed by the co-polymerization of 2-methylene-1,3,-dioxepane with dimethylphosphonate or vinylphosphonic acid. The synthesis of the present biocompatible polymer is illustrated in Scheme 1. 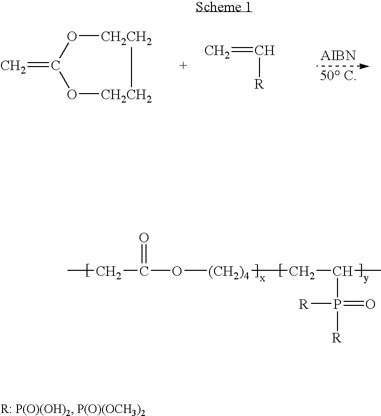
Molecular weights for the synthesized copolymer were in the range of about 146,000 as determined from gel permeation chromatography.

The copolymer may be further characterized by ¹H and ¹³C nuclear magnetic resonance, FT-IR, differential scanning calorimetry, and thermogravimetric analysis. X-ray diffraction may be used to determine polymer crystallinity, and X-ray photoelectron spectroscopy may be used to determine chemical compositions of these materials.

The biodegradable copolymer may be selectively coated with inorganic phases such as hydroxyapatite, via the simulated body fluid (SBF) approach to nucleate inorganic species of biological interest on polymer surfaces. When exposed to inorganic phases in the SBF approach, changes in copolymer molecular weight and molecular weight distributions may be monitored to characterize the physiological degradation of the copolymer. Degradation products from the copolymer may be identified as well. The protocols outlined in ASTM F2150-02e1, D6474, and ISO 10993-9 and 10993-13 may be used to conduct the degradation analyses.

In further characterizing biodegradable copolymers, appropriate amounts of the polymer may be added to 10 mL of 0.1M phosphate buffer in 22 mL glass vials to make 1, 15, and 40 mg/mL. The vials may be sealed and placed in an oven at 50° C. to accelerate degradation processes. Since the degradation products may be acidic, the degradation of the copolymers may be monitored by pH changes in the buffer solution.

The foregoing description of embodiments of the invention has been presented only for the purpose of illustration and description and is not intended to be exhaustive or to limit the invention to the precise forms disclosed. Numerous modifications and adaptations thereof will be apparent to those skilled in the art without departing from the spirit and scope of the present invention.

1. A material comprising:
a polymeric layer comprising ions implanted therein, wherein the polymeric layer is biocompatible.

2. The material of claim 1, wherein the polymeric layer comprises poly(methyl metacrylate), polyphosphazenes, polysiloxanes, ultra-high molecular weight polyethylene, polymers formed from phosphorus-containing cyclic esters, or any co-polymeric combination thereof.

3. The material of claim 1, wherein the ions implanted in the polymeric layer comprise calcium ions, phosphorus ions, magnesium ions, potassium ions, sodium ions, or any combination thereof.

4. The material of claim 1, wherein the polymeric layer has a thickness of at least about 10 μm.

5. The material of claim 1, wherein the polymeric layer has a thickness of at least about 5 μm.

6. The material of claim 1 wherein the polymeric layer has a thickness of at least about 1 μm.

7. The material of claim 1, wherein the thickness of the polymeric layer ranges from about 1 μm to about 10 μm.

8. The material of claim 1, wherein the polymeric layer is operable to promote the adhesion of osteoblast cells, fibroblast cells, and chondroblast cells.

9. A lithographic process comprising:
exposing a lithographic recording medium to an ion source to form a pattern, wherein the lithographic recording medium comprises a biocompatible polymer.

10. The lithographic process of claim 9, wherein the biocompatible polymer comprises poly(methyl methacrylate), polyphosphazenes, polysiloxanes, ultra-high molecular weight polyethylene, polymers formed from phosphorus-containing cyclic esters, or any co-polymeric combination thereof.

11. The lithographic process of claim 9, wherein the ion source comprises calcium ions, phosphorus ions, magnesium ions, potassium ions, sodium ions, or any combination thereof.

12. An apparatus comprising:
a substrate; and
a polymeric substrate coating comprising ions implanted therein, wherein the polymeric substrate coating is biocompatible.

13. The apparatus of claim 12, wherein the polymeric substrate coating comprises poly(methyl methacrylate), polyphosphazenes, polysiloxanes, ultra-high molecular weight polyethylene, polymers formed from phosphorus-containing cyclic esters, or any co-polymeric combination thereof.

14. The apparatus of claim 12, wherein the ions comprise calcium ions, phosphorus ions, magnesium ions, potassium ions, sodium ions, or any combination thereof.

15. The apparatus of claim 12, wherein the substrate comprises a metal or an alloy.

16. The apparatus of claim 15, wherein the metal comprises titanium, cobalt, chromium, molybdenum, or nickel.

17. The apparatus of claim 15, wherein the alloy comprises chromium, nickel, cobalt, molybdenum, or titanium alloys or stainless steel.

18. The apparatus of claim 12, wherein the substrate comprises a polymeric material.

19. The apparatus of claim 18, wherein the polymeric material comprises ultra-high molecular weight polyethylene, polypropylene, polycarbonate, or any combination thereof.

20. The apparatus of claim 12, wherein the substrate comprises an inner metal or alloy layer and an outer polymeric layer.

21. The apparatus of claim 20, wherein the inner metal layer comprises titanium, cobalt, chromium, molybdenum, or nickel.

22. The apparatus of claim 20, wherein the inner metal alloy layer comprises chromium, nickel, cobalt, molybdenum, or titanium alloys or stainless steel.

23. The apparatus of claim 20, wherein the outer polymeric layer comprises ultra-high molecular weight polyethylene, polypropylene, polycarbonate, or any combination thereof.

24. The apparatus as in any one of claims 12 to 23, wherein the apparatus comprises an implant.

25. A biocompatible polymer comprising:
a phosphorus component comprising at least one phosphorus containing monomer; and
a cyclic component comprising at least one cyclic containing monomer operable to undergo ring opening polymerization;
wherein the biocompatible polymer is biodegradable.

26. The biocompatible polymer of claim 25, wherein the phosphorus component comprises dimethylphosphonate (VPE), vinylphosphonic acid (VPA), or any combination thereof.

27. The biocompatible polymer of claim 25, wherein the cyclic component comprises lactones, lactams, cyclic acetals, and expoxides.

28. The biocompatible polymer of claim 27, wherein the lactones comprise 2-methylene-1,3-dioexpane (MDO).

29. The biocompatible polymer of claim 26 further comprising ions implanted therein.

30. The biocompatible polymer of claim 29, wherein the ions comprise calcium ions, phosphorus ions, magnesium ions, potassium ions, sodium ions, or any combination thereof.

31. The biocompatible polymer of claim 25, wherein the polymer is operable to promote the adhesion of osteoblast cells, fibroblast cells, and chondroblast cells.

32. A lithographic process comprising:
exposing a lithographic recording medium to an ion source wherein the lithographic recording medium comprises the biocompatible polymer of claim 26.

33. The lithographic process of claim 32, wherein the phosphorus component of the polymer comprises dimethylphosphonate (VPE), vinylphosphonic acid (VPA), or any combination thereof.

34. The lithographic process of claim 33, wherein the cyclic component comprises lactones, lactams, cyclic acetals, and expoxides.

35. An apparatus comprising:
a biocompatible polymer comprising:
a phosphorus component comprising at least one phosphorus containing monomer; and
a cyclic component comprising at least one cyclic containing monomer operable to undergo ring opening polymerization;
wherein the biocompatible polymer is biodegradable.

36. The apparatus of claim 35, wherein the phosphorus component comprises dimethylphosphonate (VPE), vinylphosphonic acid (VPA), or any combination thereof.

37. The apparatus of claim 35, wherein the cyclic component comprises lactones, lactams, cyclic acetals, expoxides, or any combination thereof.

38. The apparatus of claim 35, further comprising ions implanted in the biocompatible polymer.

39. The apparatus of claim 38, wherein the ions comprise calcium ions, phosphorus ions, magnesium ions, potassium ions, sodium ions, or any combination thereof.

40. The apparatus of claim 35, wherein the apparatus is operable to promote the adhesion of osteoblast cells, fibroblast cells, and chondroblast cells.

41. The apparatus as in any one of claims 35-40, wherein the apparatus comprises an implant.
